(19) United States
(12) Patent Application Publication
(10) Pub. No.: US 2025/0041530 A1
(43) Pub. Date: Feb. 6, 2025

(54) ASSEMBLY FOR AN INJECTION DEVICE, INJECTION DEVICE COMPRISING SUCH AN ASSEMBLY, MEDICAMENT CONTAINER FOR SUCH AN ASSEMBLY, AND METHOD FOR PRODUCING SUCH A MEDICAMENT CONTAINER

(71) Applicant: Sanofi, PARIS (FR)

(72) Inventors: Max Davis, Melbourn, Hertfordshire (GB); Thomas Mark Kemp, Melbourn, Hertfordshire (GB); Tom Lever, Melbourn, Hertfordshire (GB); Max Wehebrink, Melbourn, Hertfordshire (GB); Robbie Wilson, Melbourn, Hertfordshire (GB)

(21) Appl. No.: 18/718,749

(22) PCT Filed: Dec. 13, 2022
(86) PCT No.: PCT/EP2022/085657

§ 371 Date: Jun. 11, 2024

(30) Foreign Application Priority Data
Dec. 15, 2021 (EP) 21315279.6

(51) Int. Cl. A61M 5/32; A61M 5/34

(52) U.S. Cl. CPC A61M 5/3204; A61M 5/344; A61M 2005/3267

(57) ABSTRACT

The present disclosure concerns an assembly for an injection device, e.g. an auto-injector device, comprising: a medicament container comprising a medicament container body and a needle protruding from a distal end of the medicament container body and fluidly connected to an interior of the medicament container body, a housing comprising a first bearing surface configured to support the medicament container in a distal direction, a distancing element arranged on the needle and providing a second bearing surface configured to contact a patient's skin during use of the injection device, thereby preventing insertion of the needle into the patient's body beyond a predetermined injection depth.

### CROSS REFERENCE TO RELATED APPLICATIONS

The present application is the national stage entry of International Patent Application No. PCT/EP2022/085657, filed on Dec. 13, 2022, and claims priority to Application No. EP 21315279.6, filed on Dec. 15, 2021, the disclosures of which are incorporated herein by reference.

### BACKGROUND

Various auto-injectors for the use in adults are known. Those comprise a needle that is configured to be inserted into a patient's body for a certain injection depth before releasing a medicament. Often, this certain injection depth is unfit for use in children and/or adults with certain conditions leading to thinner skin, e.g. a low BMI. Children have for example thinner skin than adults and the underlying tissue also is of lesser thickness when compared to adults. Therefore, a reduced injection depth is desirable.

### SUMMARY

The present disclosure relates to an improved assembly for an injection device, e.g., an auto-injector device, especially regarding a reduced needle insertion depth.

In the present disclosure, an assembly for an injection device, e.g., an auto-injector device, is proposed. The assembly comprises a medicament container comprising a medicament container body and a needle protruding from a distal end of the medicament container body. Further, the needle may protrude distally beyond the housing in one or more, preferably all, states of operation, e.g. in a first state and/or in a second state (see further below). The needle is fluidly connected to or connectable to an interior of the medicament container body. The assembly further comprises a housing comprising a first bearing surface configured to support the medicament container in a distal direction. The assembly also comprises a distancing element arranged on the needle and providing a second bearing surface configured to contact a patient's skin during use of the injection device, thereby preventing insertion of the needle into the patient's body beyond a predetermined injection depth.

An injection device may be a device for injecting a medicament into a patient's body. Preferably, the injection device is a pen-type device. An auto-injector can be configured to provide a single shot of medicament, before being discarded. Preferably, the auto-injector is configured to essentially empty the medicament container in the single shot. The auto-injector may comprise an energy storage, e.g. a drive spring, for expelling the medicament from the medicament container, wherein, for example, the energy of the energy storage may be released during a single activation/shot.

The needle may be fixed to the medicament container body. The medicament container may be a syringe type medicament container. The medicament container body may comprise a tubular, e.g. cylindrical, form. The interior of the medicament container body may comprise a tubular, e.g. cylindrical form. The medicament container may comprise a stopper configured to move in the interior of the medicament container to reduce a size of an enclosed compartment and to expel medicament from the interior of the medicament container, e.g. through the needle. The stopper may seal the interior of the container body proximally. The medicament container may be a syringe with a staked needle.

The first bearing surface may limit an axial motion of the medicament container in the distal direction. Proximal movement, e.g. in a limited fashion, may be allowed. The first bearing surface may be facing partially, mainly or entirely into a proximal direction. The first bearing surface may be provided directly on the housing or on an additional part that is fixed in regards to the housing at least in a distal direction or also in proximal direction. The medicament container may comprise a bearing surface, facing or mainly facing the first bearing surface and configured to contact the first bearing surface. The bearing surface of the medicament container may be arranged on the medicament container body, e.g. on a shoulder of the medicament container, e.g. in an area where the needle is fixed to the medicament container housing, alternatively on a radial flange of the medicament container body arranged at a proximal end of the medicament container body, e.g. avers to the needle. Alternatively, the medicament container comprises an adapter part, housing the medicament container body and providing the bearing surface of the medicament container, e.g. in cases where a small diameter medicament container body is used.

The second bearing surface may comprise a surface area of at least 2 mm², e.g. of at least 3 mm².

Preferably, the distancing element is arranged directly on the needle. The injection depth may be understood as the length of the part of the needle that is inserted or insertable into the tissue through patient's skin. The predetermined injection depth may be in a range between 2 mm and 7 mm, preferably in a range between 3 mm and 5 mm. The device may prevent intra muscular injection for kids or patients with thin skin.

The distancing element may be rigidly connected to the needle. No relative axial movement may be possible between the distancing element and the needle.

The proposed assembly allows the use of medicament containers with standardized needle length also for children. Especially, a distance from the needle tip to the distancing element may be better controlled in production, e.g. may be provided with a lower tolerance, than a length of the needle, e.g. a tolerance of the length of needle from tip to medicament container body, therefore a precision of the predetermined injection depth may be increased.

Also, the distancing element may allow for an easy identification of dimensionally controlled components at certain parts during assembly and use, e.g for paediatrics.

Preferably, the distancing element is cohesively and/or adhesively bonded to the needle. The distancing element may be adhesively bonded to needle. The distancing element may be formed by material, e.g. plastics, deposited directly on the needle. A material of the distancing element may be a medical grade polymer such as PC/ACBs, Nylon, Glass Filled Nylon, and/or POM. Alternatively a metal such as Stainless Steel would also be appropriate.

According to alternative embodiments, the distancing element is mechanically locked to the needle, e.g. by pinching, clamping and/or gripping features. Therewith a secure connection can be achieved.

According to further embodiments, the needle may, for example, always be free from the distancing element in an area, e.g. a first area, between a needle tip of the needle and a point on the needle that is offset from the needle tip by the predetermined injection depth. This allows for a smooth insertion of the needle into the patient's skin. The distancing element may be rigid and/or non-deformable, e.g. when subjected to an axial force.

Alternatively the first area may be partially or completely covered by the distancing element. The distancing element may herein be configured to compress during insertion of the needle into the patient's skin, such that in a fully compressed state the needle is free from the distancing element in the first area. Nevertheless, a portion of the distancing element, e.g. a portion which is not compressible may still be fixed relative to the needle

In further embodiments, the distancing element reaches from the medicament container body at least up to the point on the needle that is offset from the needle tip by the predetermined injection depth. The distancing element may comprise a proximal surface attached to and/or abutting a distal surface of the medicament container body. Herewith, a reliable injection depth may be achieved.

Alternatively, an area, e.g. a second area, of the needle is free from the distancing element. The second area may be between the medicament container body and the point on the needle that is offset from the needle tip by the predetermined injection depth. For example, an area of needle between the distancing element and the medicament container body may be free of the distancing element. This way, material usage and/or costs may be reduced.

For example, the distancing element is configured to completely cover the needle circumferentially for the entire length of the distancing element, e.g. in all radial directions from the needle. The distancing element may be a tubular or sleeve element. Herewith, a reliably determined injection depth and/or a secure attachment may be achieved.

Alternatively, the distancing element may be configured to at least in part along its length cover one or more circumferential sections of the needle circumferentially, while leaving other circumferential sections free from the distancing element. The distancing element may, in this case, at least in part have a helical shape.

In further embodiments, the distancing element may be configured to push the medicament container into a proximal direction during use of the injection device, e.g. when the housing is moved towards the patient's skin in a state when the second bearing surface already contacts the patient's skin, such that the medicament container body, e.g. at least bearing surface of the medicament container body, is distanced to the first bearing surface, e.g. in a proximal direction and/or along the main axis. The medicament container may be pushed for a distance that is smaller than the predetermined injection depth, e.g. by at least a factor of 2, e.g. at least a factor of 5. A drive mechanism of the injection device, that is configured to act upon the medicament container to eject the medicament, e.g. at least a plunger of the drive mechanism, may be arranged in a distance to the medicament container to allow for the proximal movement of the medicament container in relation to the housing. In this way, a regular operation of the injection device and/or a reduced impact upon the patient's skin may be achieved.

In one incidence, the distancing element is, e.g. mostly and/or entirely, made of a rigid or essentially rigid material. Therewith a reliable injection depth can be achieved.

Alternatively, the distancing element may at least partially be made of a compressible and/or soft material, e.g. to reduce impact forces upon the patient's skin. A compressibility of the distancing element may herein be chosen such that after fully inserting the needle up to the distancing element and activating a medicament discharge by a drive mechanism, an impact force of the drive mechanism may cause the distancing element to compress such that the needle is further inserted into the patient's body to reach the predetermined injection depth.

Furthermore, the assembly may comprise a needle shield configured to cover at least the needle tip, and at least a portion of the distancing element and configured to be removed before use of the injection device, thereby leaving the distancing element behind on the needle. The assembly may further include a cap that is configured to be releaseably connected to the housing. The needle shield may be coupled to the cap. By the needle shield, the needle may be protected and/or sterility may be maintained more easily.

According to alternative embodiments, the needle shield may be configured to, for example, only cover a part of the needle from the tip of the needle up to the distancing element or even a smaller part.

In further embodiments, the assembly may comprise a needle cover. The needle cover may comprise a third bearing surface that protrudes further from the housing than the needle in at least a first position (e.g. of the needle cover), e.g. in a first and/or second state of operation. The needle cover may be arranged to at least radially cover a part of the needle protruding from the housing, e.g. at least in the first position of the needle cover relative to the housing. The needle cover may be coupled to and/or arranged in the housing such as to be moveable telescopically relative to the housing when a force is exerted upon it in a proximal direction. The needle cover may comprise an opening for the needle to pass through and/or for accommodating the second bearing surface of the distancing element within the opening when the needle cover is moved into the housing to a second position. The second bearing surface and the opening may be dimensioned such that the second bearing surface can be arranged within the opening. The third bearing surface may be configured for contacting the patient's skin during use of the injection device. The opening may be arranged in the third bearing surface.

The second bearing surface may in the first position of the needle cover be arranged within the needle cover. The second bearing surface may be axially aligned with the third bearing surface at least in the second position of the needle cover. Providing a needle cover may allow for a secure operation of the injection device and/or may prevent damage to the needle or a person after removal of the needle shield.

According to further embodiments, the needle cover is configured to interact with a drive mechanism of the injection device to activate and/or enable a medicament ejection, e.g. in response to movement of the needle cover from the first position to the second position relative to the housing. Activation of the medicament ejection may lead to an immediate ejection of the medicament in response to the movement of the needle cover. Enablement of the medicament ejection may release a locking mechanism that is preventing an activation, while the actual activation may be triggered by a trigger element, e.g. an activation button, to be operated by the patient or a user separately from the needle cover. This may allow for a simple and/or safe operation of the injection device.

For example, the second bearing surface may be configured to contact the patient's skin, before the needle cover interacts with the drive mechanism to activate and/or enable the medicament ejection. This may allow for a simple and/or safe operation of the injection device. A transmission of an impact of the drive mechanism onto the patient's skin may be dampened by this.

The medicament container may be prefilled with a medicament.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about-4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly (A21), Arg (B31), Arg (B32) human insulin (insulin glargine); Lys (B3), Glu (B29) human insulin (insulin glulisine); Lys (B28), Pro (B29) human insulin (insulin lispro); Asp (B28) human insulin (insulin aspart); human insulin, wherein proline in position B28 is replaced by Asp, Lys, Leu, Val or Ala and wherein in position B29 Lys may be replaced by Pro; Ala (B26) human insulin; Des (B28-B30) human insulin; Des (B27) human insulin and Des (B30) human insulin.

Examples of insulin derivatives are, for example, B29-N-myristoyl-des (B30) human insulin, Lys (B29) (N-tetradecanoyl)-des (B30) human insulin (insulin detemir, LEVEMIR®); B29-N-palmitoyl-des (B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des (B30) human insulin, B29-N-omega-carboxypentadecanoyl-gamma-L-glutamyl-des (B30) human insulin (insulin degludec, Tresiba®); B29-N—(N-lithocholyl-gamma-glutamyl)-des (B30) human insulin; B29-N-(ω-carboxyheptadecanoyl)-des (B30) human insulin and B29-N-(ω-carboxyheptadecanoyl) human insulin.

Examples of GLP-1, GLP-1 analogues and GLP-1 receptor agonists are, for example, Lixisenatide (LYXUMIA®), Exenatide (Exendin-4, BYETTA®, BYDUREON®, a 39 amino acid peptide which is produced by the salivary glands of the Gila monster), Liraglutide (VICTOZA®), Semaglutide, Taspoglutide, Albiglutide (SYNCRIA®), Dulaglutide (TRULICITY®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (KYNAMRO®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (SYNVISC®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014 (E). As described in ISO 11608-1:2014 (E), needle-based injection systems may be broadly distinguished into multi-dose container systems and single-dose (with partial or full evacuation) container systems. The container may be a replaceable container or an integrated non-replaceable container.

As further described in ISO 11608-1:2014 (E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014 (E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014 (E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

Furthermore, an injection device, e.g. an auto-injector device, comprising an earlier described assembly and a drive mechanism configured to eject medicament from the medicament container when activated, are proposed. The drive mechanism may comprise an energy storage. The energy storage may be pre-charged, e.g. during assembly of the injection device. The energy storage may be a mechanical energy storage, e.g. in form of a, e.g. compressed, spring. Alternatively, the energy storage may be of electric or pneumatic nature, e.g. compressed gas. The auto-injector device may, in comparison to other injection devices, be configured to automatically perform an ejection of a medicament, e.g. by the drive mechanism, after a medicament discharge was initiated without, for example, requiring further actions of a user of the injection device. In alternative embodiments, a discharge of the medicament may, for example, require a constant acting of the user, e.g. an exertion of a driving force, even after initiation of the medicament discharge.

Also, a medicament container comprising a distancing element for an earlier described assembly is proposed. Characteristics and features of the assembly and/or the distancing element, as described before, may also apply for the medicament container and its distancing element.

A first method for manufacturing the before mentioned medicament container is proposed, wherein the distancing element is formed directly onto the needle, before or after the needle is affixed to the medicament container body.

A second method for manufacturing the before mentioned medicament container is proposed, wherein the distancing element is a preformed, e.g. moulded or machined, part and is affixed to the needle adhesively.

According to alternative embodiments, the needle and the distancing element may be made from a single piece. The needle and the distancing element may be machined from a green body, e.g. a cylinder or a hollow cylinder, wherein the needle portion is formed by machining, e.g. turning, down the thickness of the green body further than in the portion constituting the distancing element.

### BRIEF DESCRIPTION OF THE FIGURES

FIG. 1 shows a perspective and partially transparent view of an injection device comprising an assembly in a schematic view in a second state of operation.

FIG. 2a-d shows a cross-sectional view of the assembly in first, second, third, and fourth states of operation, respectively.

### DETAILED DESCRIPTION

In the figures, identical elements, identically acting elements or elements of the same kind may be provided with the same reference numerals.

“Distal” is used herein to specify directions, ends or surfaces which are arranged or are to be arranged to face or point towards a dispensing end of the drug delivery device or components thereof and/or point away from, are to be arranged to face away from or face away from the proximal end. On the other hand, “proximal” is used to specify directions, ends or surfaces which are arranged or are to be arranged to face away from or point away from the dispensing end and/or from the distal end of the drug delivery device or components thereof. The distal end may be the end closest to the dispensing and/or furthest away from the proximal end and the proximal end may be the end furthest away from the dispensing end. A proximal surface may face away from the distal end and/or towards the proximal end. A distal surface may face towards the distal end and/or away from the proximal end. The dispensing end may be the needle end where a needle unit is or is to be mounted to the device, for example.

The distal direction may be the direction in which a tip of the needle is oriented to. A proximal direction may be a direction opposite the distal direction. The distal and proximal directions may be aligned along a main axis. The main axis may be colinear with the needle or run through the needle. Any or all moving components of the injection device may be configured to move along the main axis and/or rotate around the main axis. A radial direction may be any direction orthogonal to the main axis. The main axis may be an axis along which most of the components of the injection device are arranged and/or oriented. The main axis may be an axis of lowest moment of inertia of the injection device or at least close to such an axis.

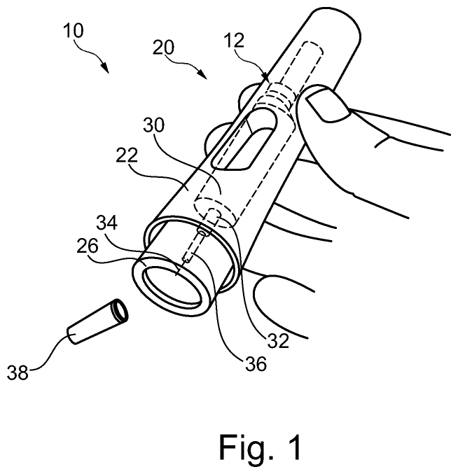

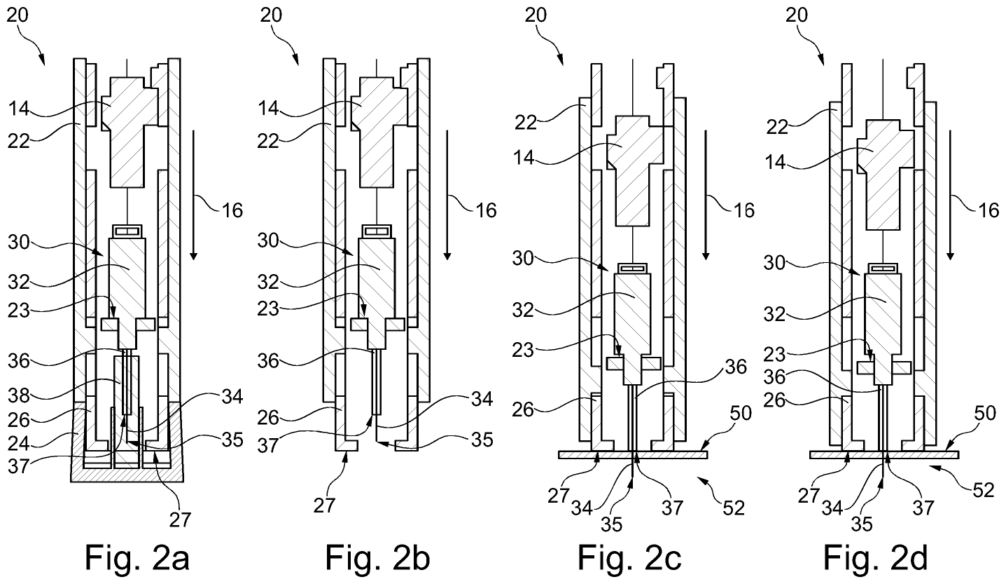

FIG. 1 shows an injection device 10. The injection device 10 is an auto-injector device. The injection device 10 is a pen-type device. The injection device comprises an assembly 20 (also compare FIGS. 2a-d) and a drive mechanism 12. The drive mechanism 12 may be part of the assembly. Details of the drive mechanism 12 may be found in the PCT patent application WO 2015/004052 A, the disclosure of which is herewith incorporated in this application. The drive mechanism may be the same or similar to the one disclosed in WO 2015/004052 A.

The assembly 20 for the injection device 10 comprises a medicament container 30. The medicament container 30 comprises a medicament container body 32 and a needle 34 protruding from the medicament container body 32. The Needle 34 is fluidly connected to an interior of the medicament container body 32. The medicament container 30 is prefilled with a medicament. The drive mechanism 12 is configured to eject medicament from the medicament container 30 when activated. The drive mechanism 12 comprises an energy storage (not shown) and a plunger 14, wherein the energy storage is configured to drive the plunger towards and/or into the medicament container 30, e.g. in a distal direction 16. The plunger 14 is configured to interact with the medicament container 30 to eject medicament from the medicament container 30. The energy storage may be a compressed spring, e.g. arranged within the plunger 14.

The assembly 20 further comprises a housing 22. The housing comprises a first bearing surface 23 configured to support the medicament container 30 in the distal direction 16.

The assembly 20 further comprises a distancing element 36 arranged on the needle 34. The distancing element 36 provides a second bearing surface 37 configured to contact a patient's skin 50 during use of the injection device 10, thereby preventing insertion of the needle 34 into the patient's body 52 beyond a predetermined injection depth.

The distancing element 36 is cohesively bonded to the needle 34. The needle 34 is free from the distancing element 36 in an area between a needle tip 35 of the needle and a point on the needle that is offset from the needle tip by the predetermined injection depth. The distancing element 36 reaches from the medicament container body 32 at least till the point on the needle 34 that is offset from the needle tip 35 by the predetermined injection depth. The second bearing surface 37 touches the point on the needle 34 that is offset from the needle tip 35 by the predetermined injection depth. The distancing element 36 completely covers the needle 34 circumferentially for the entire length of the distancing element 36. The distancing element 36 is a tubular and/or cylindrical element. The distancing element 36 surrounds the needle 34 radially. The distancing element 36 is made from an essentially rigid material. The distancing element 36 is made from a polymer.

The assembly 20 further comprises a needle shield 38 configured to cover at least the needle tip 35, the needle 34 and the distancing element 36. The needle shield 38 is configured to be removed before use of the injection device 10, thereby leaving the distancing element 36 behind on the needle 34. The assembly 20 further comprises a cap 24. The cap 24 is releaseably coupled to the housing 22. The cap 24 mechanically locked to the needle shield 38 such that the needle shield 38 is removed from the needle 34, when the cap is removed from the housing 22. In a first state of operation (compare FIG. 2a) the cap 24 may still be attached. In a second state of operation the cap 24 and needle shield 38 are removed and the injection device 10 is ready for use (compare FIG. 2b).

The assembly 20 further comprises a needle cover 26 for at least radially covering a part of the needle 34 protruding from the housing 22 at least in a first position of the needle cover 26, e.g. in the first and second state of operation (FIGS. 2a and 2b). The needle cover 26 comprises a cylindrical shape at least in the area where the needle 34 protrudes from the housing 22. The needle cover 26 comprises a third bearing surface 27. The third bearing surface is arranged on a distal end of the needle cover 26. The needle cover 26 may comprise a section that is formed on a distal end of the needle cover 26 and that extends radially inwards.

The needle cover 26 is arranged in the housing 22 such as to move telescopically in the housing 22 when a force is exerted upon it in a proximal direction (avers to the distal direction 14), e.g. by pressing the third bearing surface 27 against the patient's skin 50. The needle cover comprises a distal opening for the needle 34 and the distancing element 36 to pass through when the needle cover 26 is moved into the housing 22 to a second position, e.g. in a third state of operation (compare FIG. 2c).

The distancing element 36 is configured to push the medicament container 20 into the proximal direction during use of the injection device 10, e.g. when the needle cover 26 is moved from the first position into the second position, such that the medicament container body 32 is proximally distanced to the first bearing surface 23.

The needle cover 26 is configured to interact with the drive mechanism 12 of the injection device 10 to activate and/or enable a medicament ejection. When the needle cover 26 reaches the second position, the needle cover 26 loses engagement with the plunger 14. In one embodiment, the energy storage may configured to release stored energy to drive the plunger 14 into the distal direction to interact with the medicament container 20 to release the medicament as soon as the needle cover 26 loses engagement with the plunger 14. In another embodiment, after the needle cover 26 loses engagement with the plunger 14, an additional user action, e.g. a button press, may be needed to release the stored energy and to move the plunger 14.

The second bearing surface 35 is configured to contact the patient's skin 50, before the needle cover 26 interacts with the drive mechanism 12 to activate and/or enable the medicament ejection.

In a fourth state of operation (FIG. 2d), a proximal bearing surface of the needle cover 26 may contact a distal bearing surface of the housing 22 such that a further movement of the needle cove 26 into the housing 22 is hindered. The fourth and third state of operation may be the same state of operation. In the fourth state of operation, the medicament container 20 is distanced proximally to the first bearing surface further than in the third state of operation.

The medicament container 20 may be produced, by forming the distancing element 36 directly onto the needle 34, before or after the needle is affixed to the medicament container body 32.

The distancing element 36 may be formed from molten material that is applied to the needle 34 and solidifies there.

Alternatively, the medicament container 20 may be produced by providing the needle 34 and the distancing element 36. The needle 34 may be inserted into a hollow channel of the distancing element 36. The distancing element 36 may mantle/line/sheath the needle 34. An adhesive may be added to the needle before inserting it into the distancing element 36 or the adhesive may be added between the needle 34 and the distancing element 36 after the needle was inserted into the distancing element. Alternatively, the distancing element 36 may be made from a thermoplastic material that is heated after the needle 36 is inserted in order to attach the distancing element 36 to the needle 34 adhesively. The distancing element 36 may be adhesively connected to the needle 34 before or after the needle 34 is affixed to the medicament container body 32.

The assembly 20 and/or the injection device 10 may be provided by using a further assembly, wherein the further assembly comprises a medicament container 30, wherein the medicament container 30 comprises a medicament container body 32 and a needle 34 protruding from the medicament container body 32 and fluidly connected to an interior of the medicament container body 32, wherein during use of the injection device 10, the needle 34 is configured to be inserted into a patient's body 52 up to a further predetermined injection depth, wherein a distancing element 36 is arranged on the needle 34 that provides a second bearing surface 37 configured to contact the patient's skin 50 during use of the injection device 10, thereby preventing insertion of the needle 34 into the patient's body 52 beyond a predetermined injection depth that is smaller than the further predetermined injection depth. The further assembly may comprise the housing 22, the needle cover 26, the cap 24 and/or the drive mechanism 12.

### REFERENCE NUMERALS

10 injection device
12 drive mechanism
14 plunger
16 distal direction
20 assembly
22 housing
23 bearing surface
24 cap
26 needle cover
27 bearing surface
30 medicament container
32 medicament container body
34 needle
35 needle tip
36 distancing element
37 bearing surface
38 needle shield
50 patient's skin
52

1-17. (canceled)

18. An assembly for an injection device, comprising:
a medicament container, comprising:
a medicament container body; and
a needle protruding from a distal end of the medicament container body and fluidly connected to an interior of the medicament container body;
a housing comprising a first bearing surface configured to support the medicament container in a distal direction; and
a distancing element arranged on the needle and providing a second bearing surface configured to contact a patient's skin during use of the injection device, thereby preventing insertion of the needle into the patient beyond a predetermined injection depth.

19. The assembly according to claim 18, wherein the distancing element is cohesively bonded to the needle.

20. The assembly according to claim 18, wherein the needle is free from the distancing element in an area between a needle tip of the needle and a point on the needle that is offset from the needle tip by the predetermined injection depth.

21. The assembly according to claim 18, wherein the distancing element extends from the medicament container body at least to a point on the needle that is offset from a needle tip by the predetermined injection depth.

22. The assembly according to claim 18, wherein the distancing element is configured to completely cover the needle circumferentially for an entire length of the distancing element.

23. The assembly according to claim 18, wherein the distancing element is configured to push the medicament container into a proximal direction during use of the injection device, such that the medicament container body is spaced from the first bearing surface.

24. The assembly according to claim 18, wherein the distancing element is made from a rigid material.

25. The assembly according to claim 18, further comprising a needle shield configured to cover at least a needle tip and at least a portion of the distancing element and configured to be removed before use of the injection device, thereby leaving the distancing element behind on the needle.

26. The assembly according to claim 18, further comprising a needle cover providing a third bearing surface that protrudes further from the housing than a needle tip at least in a first position of the needle cover, wherein the needle cover is arranged in the housing such as to move telescopically in the housing when a force is exerted upon the needle cover in a proximal direction, and wherein the needle cover comprises an opening for the needle, the opening being configured to allow the needle to pass through the opening and to accommodate the distancing element when the needle cover is moved into the housing to a second position, wherein the second bearing surface and the opening are dimensioned such that the second bearing surface can be arranged within the opening.

27. The assembly according to claim 26, wherein the needle cover is configured to interact with a drive mechanism of the injection device to activate and/or enable a medicament ejection.

28. The assembly according to claim 27, wherein the second bearing surface is configured to contact the patient's skin, before the needle cover interacts with the drive mechanism to activate and/or enable the medicament ejection.

29. The assembly according to claim 18, wherein the medicament container contains a medicament.

30. The assembly according to claim 18, wherein the needle protrudes distally beyond the housing in a first state of operation.

31. An injection device comprising:
an assembly, comprising:
a medicament container, comprising:
a medicament container body; and
a needle protruding from a distal end of the medicament container body and fluidly connected to an interior of the medicament container body; and
a housing comprising a first bearing surface configured to support the medicament container in a distal direction;
a distancing element arranged on the needle and providing a second bearing surface configured to contact a patient's skin during use of the injection device, thereby preventing insertion of the needle into the patient's skin beyond a predetermined injection depth; and
a drive mechanism configured to eject medicament from the medicament container when activated.

32. The injection device according to claim 31, wherein the medicament container contains a medicament.

33. A method of delivering a medicament from an injection device, the method comprising delivering the medicament from the injection device according to claim 32.

34. A method of manufacturing a medicament container for an injection device, the method comprising:
fluidly connecting an interior of a medicament container body of a medicament container to a needle; and
arranging a distancing element on the needle, wherein the distancing element is configured to provide a bearing surface for contacting a patient's skin during use of the medicament container in an injection device, thereby preventing insertion of the needle into the patient beyond a predetermined injection depth.

35. The method according to claim 34, wherein the distancing element is formed directly onto the needle.

36. The method according to claim 34, wherein the distancing element is a preformed part and is affixed to the needle adhesively.

37. The method according to claim 34, comprising filling the medicament container with a medicament.
